(19) United States
(12) Patent Application Publication
(10) Pub. No.: US 2026/0207366 A1
(43) Pub. Date: Jul. 23, 2026

(54) REVERSIBLY CLOSABLE LIMB ORTHOSIS OR LIMB BANDAGE OR SPORTS AID DEVICE

(71) Applicant: BAUERFEIND AG, Zeulenroda-Triebes (DE)

(72) Inventors: Gerald STIER, Zeulenroda-Triebes (DE); Sandro HEBENSTREIT, Zeulenroda-Triebes (DE); Beatrix BAUERFEIND-JOHNSON, Zeulenroda-Triebes (DE)

(21) Appl. No.: 19/136,858

(22) PCT Filed: Dec. 11, 2023
(86) PCT No.: PCT/EP2023/085097

§ 371 Date: Jun. 6, 2025

(30) Foreign Application Priority Data
Dec. 13, 2022 (DE) 10 2022 213 570.9

(51) Int. Cl. A61F 5/01

(52) U.S. Cl. CPC A61F 5/0125; A61F 5/013; A61F 2005/0167; A61F 2005/0179

(57) ABSTRACT

The present invention relates to a limb orthosis, a limb bandage or a sports aid device comprising a tensioning system consisting of a first tensioning sub-system and a second tensioning sub-system, wherein the first tensioning sub-system and the second tensioning sub-system are each reversibly closable.

The present invention relates to a limb orthosis or limb bandage or a sports aid device comprising a tensioning system consisting of a first tensioning sub-system and a second tensioning sub-system, wherein the first tensioning sub-system and the second tensioning sub-system are each reversibly closable.

Orthoses and bandages for limbs, also known as limb orthoses or bandages, particularly knee orthoses for passive and active stabilization of the knee joint, but also elbow orthoses, are known from the prior art. Such knee orthoses often comprise a support element in the form of a bandage on which stabilizing elements such as straps, bars or joint splints are mounted, as shown, for example, in DE 200 05 366 U1.

Such knee orthoses are used, for example, in cases of knee arthrosis or after injury to the medial meniscus in order to relieve and stabilize the affected area. Knee orthoses often comprise straps with which the knee orthosis can be adjusted, i.e., adapted to the shape of the leg and/or the area of action. Specifically guided tensioning systems are known, for example, from EP 2 612 626 A2, DE 40 136 93 A1, DE 10 2017 220 968 A1, WO 1996/16615 A1, and DE 198 44 545 A1.

These tensioning systems require the wearer, particularly the patient, to step into the tensioning system with the limb, particularly the leg, when putting on the orthosis or bandage and to step out when taking it off, which can be complicated and, in particular, uncomfortable and dangerous for patients.

The problem underlying the present invention is to provide a limb orthosis or bandage, particularly a knee orthosis or bandage, which overcomes the problems of the prior art and, in particular, allows the orthosis or bandage to be put on and taken off more easily and safely.

The present invention solves the underlying technical problem by means of a limb orthosis or bandage or a sports aid device according to claim 1.

The present invention solves the underlying technical problem by means of a limb orthosis or limb bandage or a sports aid device comprising a tensioning system consisting of a first tensioning sub-system and a second tensioning sub-system, characterized in that the first tensioning sub-system and the second tensioning sub-system are each reversibly closable.

All of the following embodiments of a limb orthosis or limb bandage according to the invention are also disclosed for sports aid devices according to the invention.

It was found that it is advantageous to provide a fully functional tensioning system that can be closed and opened reversibly. This allows the wearer, particularly the patient, to advantageously place the tensioning system around the limb, particularly around the leg, and close it when putting on the orthosis or bandage, so that it is not necessary to step into the tensioning system with the leg or insert the arm. The same applies when removing the orthosis or bandage, in which case the tensioning system can be opened again and thus the tensioning system can be removed without the wearer, in particular the patient, having to pull the limb out of the tensioning system.

In a preferred embodiment, in the applied limb orthosis or bandage, the first tensioning sub-system and/or the second tensioning sub-system of the tensioning system are guided around the limb, in particular a leg, in such a way that the first tensioning sub-system and/or the second tensioning sub-system of the tensioning system each enclose the limb.

According to the invention, it is particularly provided that the first tensioning sub-system is assigned to the upper part of the limb, i.e. the thigh or upper arm, and the second tensioning sub-system is assigned to the lower part of the limb, i.e. the lower thigh or forearm, so that the knee or elbow is positioned between the two tensioning sub-systems in the worn state.

In a preferred embodiment, the first tensioning sub-system and the second tensioning sub-system are formed from a continuous tensioning element. It is therefore advantageous to form the limb orthosis or bandage according to the invention with only a single tensioning element.

Alternatively, it may of course also be provided that the first tensioning sub-system is formed from a first tensioning element and the second tensioning sub-system is formed from a second tensioning element.

In a preferred embodiment, the at least one tensioning element is a strap, a flat band, a rope, a wire or a Bowden cable. In a preferred embodiment, the at least one tensioning element is a rope, a wire or a Bowden cable.

The tensioning element can be flexible or inflexible or partially flexible. In a preferred embodiment, the at least one tensioning element is inelastic.

In a preferred embodiment, the at least one tensioning element comprises a rope or a wire, wherein the rope or the wire runs at least partially in a sheathing, wherein the sheathing is displaceably mounted on the limb orthosis or bandage.

The Bowden cable and thus also the rope can be fastened to padding at least in sections. The padding, which may also be designed as a strap or belt, serves to advantageously increase the wearing comfort for the patient and is intended to prevent the rope from cutting into the limb. The padding may be elastic or inelastic. Padding comprising only partially elastic areas is also possible.

The at least one tensioning element can also be connected to the padding via rotatably mounted fastening elements. In the preferred embodiment, the padding and the entire rope guide are slightly elastic. In a preferred embodiment, the limb orthosis or bandage comprises rotatably mounted deflection guides in which the at least one tensioning element is deflected.

In a preferred embodiment, the first tensioning sub-system is reversibly closable by a first coupling device and the second tensioning sub-system is reversibly closable by a second coupling device.

In a preferred embodiment, the first tensioning sub-system and the second tensioning sub-system are each reversibly closable by a coupling device, wherein each coupling device comprises a first coupling element and a second coupling element, wherein the first coupling element is reversibly connectable to the second coupling element via a latching system, wherein the first coupling element comprises a first guide for a tensioning element and the second coupling element comprises a second guide for a tensioning element, wherein the tensioning element runs in the first guide and the tensioning element runs in the second guide and wherein, in the connected state of the two coupling elements, the first guide and the second guide cross each other at least once, particularly twice.

By crossing the guides at least once, particularly twice, the tensioning elements located therein also cross each other. If the two coupling elements are connected to each other, for example to close the orthosis, the two tensioning elements guided therein cross each other in two different planes. Crossing the tensioning elements has the advantage that the coupling elements can be realized in a relatively small space and friction is minimized by this design. This also leads to better force distribution within the entire tensioning system.

A preferred double crossing of the guides occurs, for example, when the two guides are curved in opposite directions and overlap. A single crossing of the guides occurs, for example, when the two guides are curved in opposite directions and only overlap at their tips, i.e., at the apexes of the curvature.

It is preferred that the first guide comprises a curvature, in particular is horseshoe-shaped, and that the second guide comprises a curvature, in particular is horseshoe-shaped. Preferably, the two horseshoe-shaped guides overlap in the connected state of the two coupling elements. Preferably, the two ends of the first horseshoe-shaped guide point away from the second horseshoe-shaped guide and the two ends of the second horseshoe-shaped guide point away from the first horseshoe-shaped guide.

The guides can be designed as tunnels or channels, for example, or as interrupted tunnels or channels. The tensioning elements can cross each other without coming into contact thanks to the guides.

In a preferred embodiment, the first guide is positioned in the peripheral area of the first coupling element and the second guide is positioned in the peripheral area of the second coupling element.

In a preferred embodiment, the first coupling element comprises a front side and a back side and comprises a horseshoe shape with a concave inner edge, wherein the second coupling element comprises a front side and a back side and comprises a horseshoe shape with a concave inner edge, wherein the first coupling element comprises a first latching element in the area of the tip of the horseshoe shape on the front side and the second coupling element comprises a second latching element in the area of the tip of the horseshoe shape on the back side, and wherein the second horseshoe-shaped coupling element comprises a first bar element spanning the concave inner edge of the second coupling element, wherein, in the connected state of the two coupling elements, the first latching element of the first coupling element engages in the first bar element of the second coupling element and the second latching element of the second coupling element engages in the concave inner edge of the first coupling element.

It is preferred that the first coupling element and the second coupling element are displaceable or rotatable relative to each other in the connected state of the two coupling elements. This has the advantage that the tensioning elements are also displaceable relative to each other.

It is preferred that the second coupling element comprises a second bar element spanning the concave inner edge of the second coupling element, which presses the first bar element against the first latching element in the connected state of the two coupling elements. Preferably, the second bar element comprises an oblique flattening in its central region, which can slide under the first bar, particularly when bent, and lifts it up or otherwise detaches it from the latching element.

It is preferred that the first coupling element and the second coupling element are made of plastic.

It is preferred that the first guide and/or the second guide are designed as handle elements.

A coupling device as described is also preferred, wherein the first coupling element is reversibly connected to the second coupling element via the latching system and the coupling device is opened by pressing the second horseshoe-shaped coupling element together at the arms of the horseshoe-shaped coupling element, whereby the second bar element spanning the concave inner edge of the second coupling element is preferably pushed toward the tip of the second horseshoe-shaped coupling element and/or toward the first coupling element, and whereby the second bar element spanning the concave inner edge of the second coupling element preferably lifts the first bar element out of the first latching element, so that the two coupling elements are preferably pushed against each other and can thus be separated from each other.

In a preferred embodiment, the limb orthosis or bandage comprises a connection element to which the at least one tensioning element, in particular the single tensioning element or the first tensioning element and/or the second tensioning element, are fastened.

In a preferred embodiment, the at least one tensioning element is fastened with both ends to the connection element. In a preferred embodiment, the one, i.e. the only tensioning element, is fastened with both ends to the connection element. If there are several tensioning elements, it may also be provided that these are fastened with only one end each to the connection element.

In a preferred embodiment, the connection element comprises a tension control element for tensioning the at least one tensioning element. In a preferred embodiment, the connection element is a tension control element for tensioning the at least one tensioning element.

It is therefore advantageously provided that the two tensioning sub-systems can be tensioned together or separately, so that, on the one hand, they enable the orthosis or bandage for limbs to fit securely on the limb, in particular the knee orthosis or bandage on the leg, and, on the other hand, they enable good positioning and a good fit of a preferred joint splint.

In a preferred embodiment, the tension control element is designed as a rotary knob with which the tensioning element can be tensioned or the two tensioning elements can be tensioned simultaneously. The tensioning elements are therefore preferably tensioned by turning a knob, for example by winding ropes that form the tensioning elements by means of the rotary knob. It is therefore preferably provided that the two tensioning sub-systems are tensioned simultaneously by a tension control element so that the two tensioning sub-systems have the same tension force and thus the two tensioning sub-systems are combined to form a single tensioning system and only one working step is necessary to tension both tensioning sub-systems.

Of course, the tension control element is designed so that the tensioning systems can also be released again. For example, different tension levels can be set by means of a detent in the rotary knob, so that a specific force setting can be achieved in an advantageous manner.

In a preferred embodiment, the at least one tensioning element is fastened with both ends to the tension control element. However, it can also be fastened with only one end to the tension control element. The second end can then be fastened to any point of the orthosis.

A first tensioning element for the first tensioning sub-system and a second tensioning element for the second tensioning sub-system may also be provided, wherein each of the two tensioning elements is fastened with one end to the tension control element and with the second end to any point of the orthosis or bandage, for example to a joint splint.

In a preferred and advantageous embodiment, the tensioning system is guided in such a way that the first tensioning sub-system crosses in a first crossing area and the second tensioning sub-system crosses in a second crossing area. Preferably, the first crossing area is formed by the first coupling device and the second crossing area by the second coupling device.

Preferably, the first and/or second crossing area is located on the inside of the limb in the worn state. Preferably, the first and/or second coupling device is located on the inside of the limb in the worn state. Alternatively, the crossing areas and/or the coupling devices may be located on the outside of the limb or at the rear or at the front.

It is therefore preferred that, when the limb orthosis or bandage is applied, the first tensioning sub-system and/or the second tensioning sub-system of the tensioning system are guided around the limb, in particular a leg, in such a way that the first tensioning sub-system and/or the second tensioning sub-system of the tensioning system encloses the limb and crosses itself once.

The present invention also relates to a limb orthosis or bandage comprising a tensioning system consisting of a first tensioning sub-system and a second tensioning sub-system, wherein the two tensioning sub-systems are preferably formed from a single tensioning element and wherein the tensioning system is guided in such a way that the first tensioning sub-system crosses itself in a first crossing area and that the second tensioning sub-system crosses itself in a second crossing area, wherein the first and/or the second crossing area lies/lie on the inside of the limb in the worn state and wherein preferably both ends of the tensioning element are connected to a tension control element. This limb orthosis or bandage may be reversibly closable or non-reversibly closable.

A preferred guide for the tensioning element in a limb orthosis according to the invention with only one tensioning element is as follows in the worn state:

The tensioning element starts with the first end approximately at the level of the knee or elbow at the tension control element and runs upward over the thigh or upper arm. The tensioning element then runs to one side of the limb, particularly to the inside of the limb, where it is deflected upwards by the first coupling element of the first coupling system of the first tensioning sub-system, whereupon the tensioning element runs again over the thigh or upper arm. The tensioning element then slides over the front area of the thigh or upper arm to the other side of the limb, particularly the outside of the limb, and runs over the back of the thigh or upper arm to the first side, particularly the inside of the thigh or upper arm. There, the tensioning element is deflected downward in the second coupling element of the first coupling system and guided further toward the rear thigh or upper arm. Due to the closed first coupling system, the tensioning element thus crosses itself twice. The tensioning element runs slightly obliquely downwards on the rear thigh or upper arm to the second side, particularly the outside of the limb, and is guided there vertically downwards under the joint, i.e. the knee or elbow, to the lower thigh or forearm and thus to the second tensioning sub-system. There, the tensioning element runs over the back of the lower thigh or forearm to the first side, particularly the inside of the lower thigh or forearm. There, the tensioning element is deflected downward in the second coupling element of the second coupling system of the second tensioning sub-system and guided further in the direction of the rear lower thigh or forearm. The tensioning element runs slightly obliquely on the rear lower thigh or forearm to the second side, particularly the outside of the limb, and is guided there in the direction of the front lower thigh or forearm and from there further to the first side, particularly the inside of the lower thigh or forearm. There, the tensioning element is deflected upwards by the first coupling element of the second coupling system of the second tensioning sub-system, whereupon the tensioning element runs again over the front side of the lower thigh or forearm and ends again with the second end at the tension control element. With the tension control element, both ends of the tensioning element can thus be tensioned, for example by rolling them up.

The tension control element is preferably located on the outside of the limb, particularly if the coupling systems are located on the inside. Alternatively, the tension control element is located on the inside of the limb, particularly if the coupling systems are located on the outside.

In a preferred embodiment, a limb orthosis or bandage according to the invention comprises at least one joint splint, preferably two joint splints. In the worn state, the two joint splints are preferably positioned laterally, i.e., one on the outside of the limb and one on the inside of the limb. Preferably, the limb orthosis or bandage comprises a joint splint which, in the worn state, is positioned on the outside of the limb. Alternatively, the limb orthosis or bandage comprises a joint splint which, in the worn state, is positioned on the inside of the limb. When using two joint splints, it is preferred that one of the two joint splints, in particular the joint splint positioned on the outside of the limb, is connected to the tension control element. Preferably, a second joint splint, in particular the joint splint on the inside of the limb, is connected to one of the two coupling elements of the first tensioning sub-element and to one of the two coupling elements of the second tensioning sub-element.

Joint splints are usually designed so that the knee can be adjusted not only in the sagittal plane but also in the frontal plane. In the frontal plane, these are adjustable hinge joints that are located as close as possible to the apex of the knee joint or elbow joint in order to relieve the inner compartment of the femorotibial joint or elbow joint. The circularly arranged straps shown in the prior art act as counter bearings above and below the knee joint on the opposite side of the joint splint. Such joint splints are known, for example, from WO 2007/145504 A1 or WO 2003/103547 A1.

In a preferred embodiment, the at least one joint splint comprises a first splint section, a second splint section and a third splint section, wherein the first splint section and the second splint section are each connected to the third splint section by a joint.

In a preferred embodiment, the splint sections are designed to be elastic. In a preferred embodiment, the third splint section is formed from a spring element, in particular a leaf spring. The other two splint sections can also be formed from leaf springs.

In a preferred embodiment, the joints are monocentric joints. The two monocentric joints above and below the third, middle splint section of a joint splint together form a duo-centric, which advantageously guides the knee so that flexion of the knee generates less stress in the knee joint. During flexion, the center of rotation can change several times from one pivot point to another. In combination with elastic splints, particularly the leaf spring, the duo-centric ensures that the orthosis or bandage does not shift sideways against the limb axis, particularly the leg axis, during flexion.

The formation of the third splint section from a spring element, particularly from a leaf spring, for example made of spring steel, means that the first splint section and the second splint section not only rotate in one axis, but are also movable in an axis rotated by 90°, preferably in the area in which the coupling element makes the central force application point of the three-point system created by the tensioning system flexible. In addition, the two joints connecting the first splint section and the second splint section with the third splint section enable the joint splint as such to adapt much better to the contours of the body. The spring action of the third splint section also has the advantage that the orthosis or bandage for a limb, particularly a knee orthosis, can give way slightly during extreme movements, thereby warning the user against excessive incorrect leg movement.

In a preferred embodiment, the connection element is fastened to the third splint section of the at least one joint splint, in particular one of the two joint splints.

In the orthosis or bandage for limbs, particularly knee orthosis or knee bandage, a support element may additionally be provided, for example a bandage base element, particularly a knitted or woven fabric, but the support element may also be dispensed with due to the arrangement of the tensioning system according to the invention.

The limb orthosis or bandage may additionally comprise a bandage base body. Suitable bandage base bodies, in particular tubular bandage base bodies, are known to the person skilled in the art. The bandage base body is preferably not permanently connected to the remaining limb orthosis or bandage, but rather not connected at all or connected in a reversible manner to the remaining limb orthosis or bandage. This allows the bandage base body to be pulled over the limb in the conventional manner and the remaining limb orthosis or bandage to be applied in the advantageous manner described above. In an alternative embodiment, the bandage base body may also be firmly and permanently connected to the remaining limb orthosis or bandage, for example if the bandage base body can also be opened, for example by means of a zipper, a Velcro fastener or buttons, and thus the bandage base body can also be placed around the limb when putting it on and then closed, and the wearer does not have to put the limb through the bandage base body.

Of course, it may also be provided that no bandage base body is used, so that the limb orthosis or bandage is worn directly on the skin or on an item of clothing. This allows the orthosis or bandage to be adapted to the patient's course of therapy.

Whether a product is classified as an orthosis or a bandage depends on whether the product comprises a bandage-like knitted or woven fabric and/or how heavily reinforced the product is.

In a preferred embodiment, the limb orthosis or bandage is a knee orthosis or bandage.

In a preferred embodiment, the limb orthosis is a knee orthosis. The simpler application and removal of the orthosis is particularly advantageous in the case of a knee orthosis. However, the advantages are also present in an elbow orthosis, so that the limb orthosis can also be an elbow orthosis.

In a preferred embodiment, the limb bandage is a knee bandage. The simpler application and removal of the bandage is particularly advantageous in the case of a knee bandage. However, the advantages are also present in an elbow bandage, so that the limb bandage can also be an elbow bandage.

The limb orthosis or bandage according to the invention can also be used as a sports aid device. In an alternative embodiment, the limb orthosis or bandage according to the invention is a sports aid device. The invention also relates to a sports aid device according to claim 1. Preferred embodiments of the sports aid device according to the invention result from the embodiments for the limb orthosis and limb bandage according to the invention.

The present invention also relates to the use of an orthosis or bandage according to the invention for treating disorders of the limbs. The present invention also relates to the use of a knee orthosis or knee bandage according to the invention for treating knee disorders, in particular knee joint arthrosis, also known as gonarthrosis. Gonarthrosis refers to wear and tear of the cartilaginous joint surfaces of the knee joint. If the medial or inner compartment of the femorotibial joint is affected, this is referred to as medial gonarthrosis. If the patient also suffers from bow legs, varus gonarthrosis is present. If the lateral or outer compartment of the femorotibial joint is affected, this is referred to as lateral gonarthrosis. If the patient also has knock knees, this is referred to as valgus gonarthrosis. The knee orthosis or bandage according to the invention is preferred for medial gonarthrosis, particularly for relieving the inner compartment of the femorotibial joint and for reducing pain.

The present invention also relates to a treatment method for treating gonarthrosis, particularly medial gonarthrosis, in which a knee orthosis or bandage according to the invention is applied to the leg of a patient.

The present invention also relates to a treatment method for treating limb disorders, in which a limb orthosis or bandage according to the invention is applied to the limb of a patient.

When this description refers to the position of an element of the orthosis or bandage in relation to a part of the body, this refers to the orthosis or bandage in the state in which it is worn, i.e. when the orthosis or bandage is applied to the limb.

The invention is explained in more detail using an example and the figures, wherein these are not to be understood as limiting.

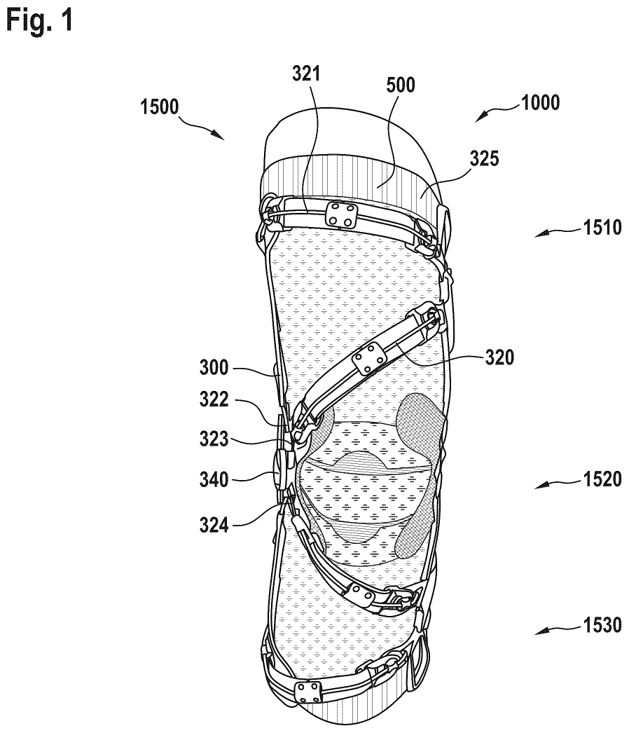

FIG. 1 shows a limb orthosis according to the invention as a knee orthosis,

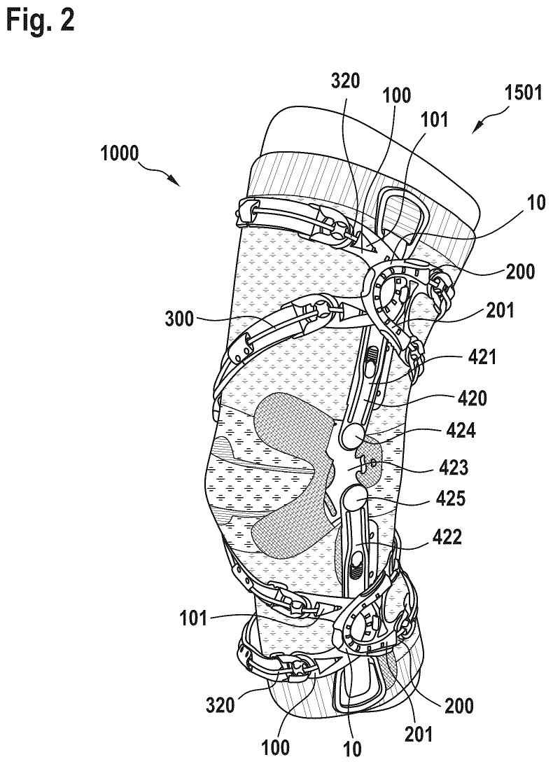

FIG. 2 to FIG. 7 show different perspectives of the knee orthosis from FIG. 1,

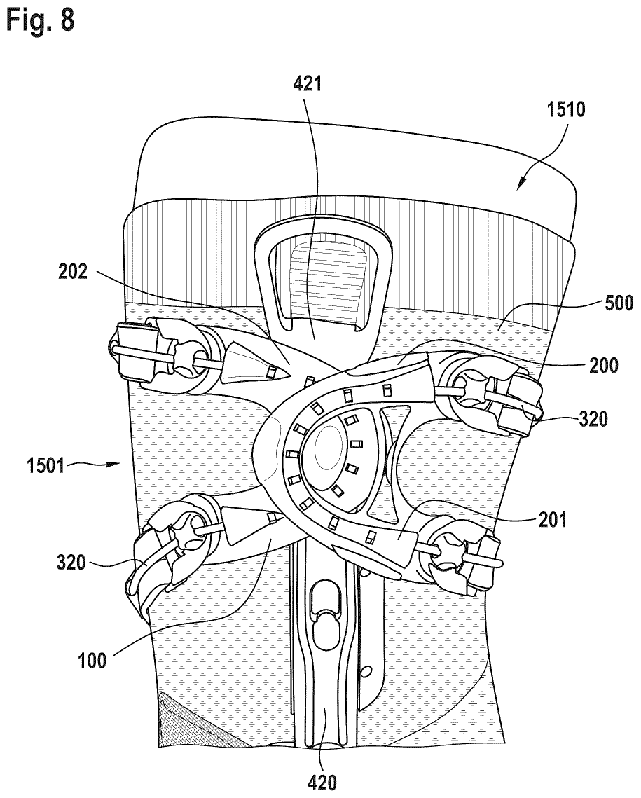

FIGS. 8 to 11 show detailed views of the knee orthosis from FIG. 1,

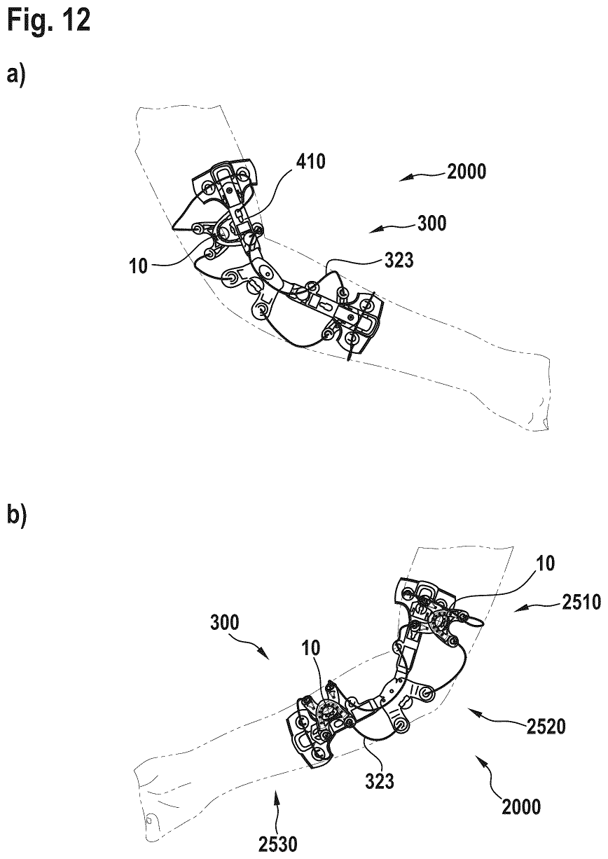

FIG. 12 shows a limb orthosis according to the invention as an elbow orthosis,

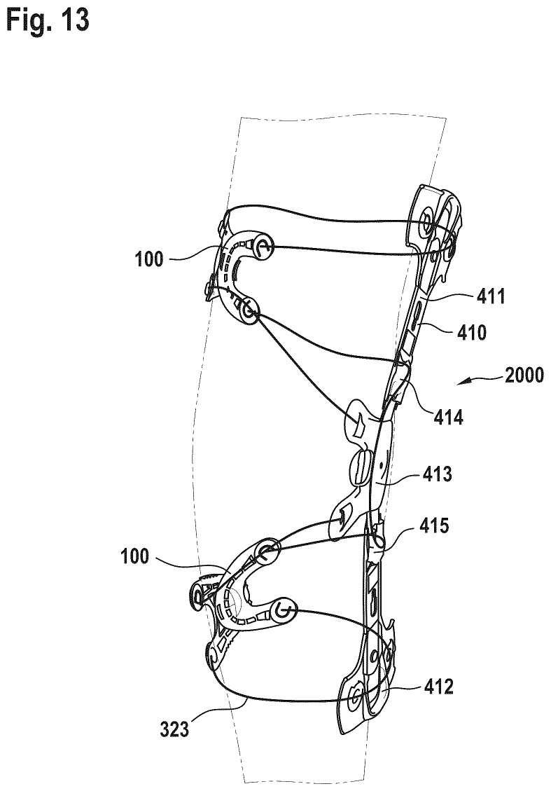

FIG. 13 shows the elbow orthosis from FIG. 12,

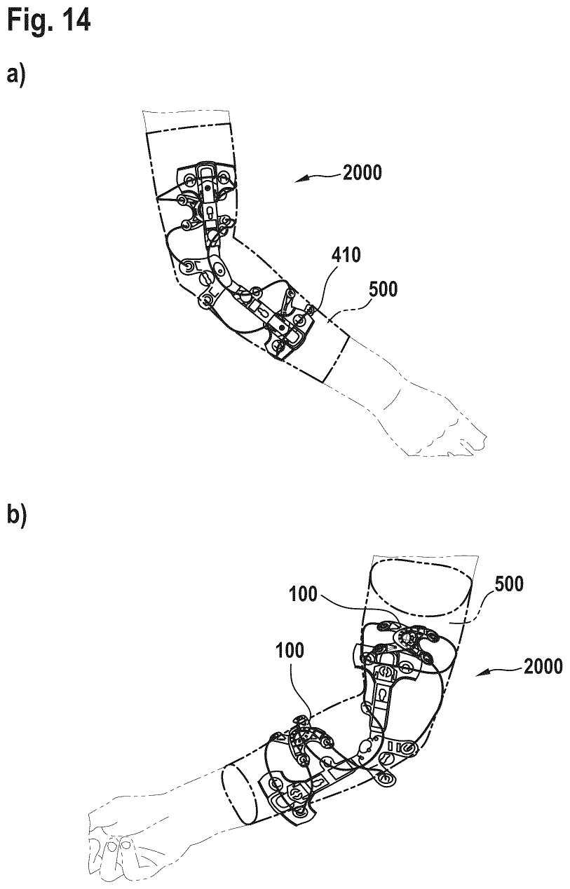

FIG. 14 shows a limb orthosis according to the invention as an elbow orthosis with bandage,

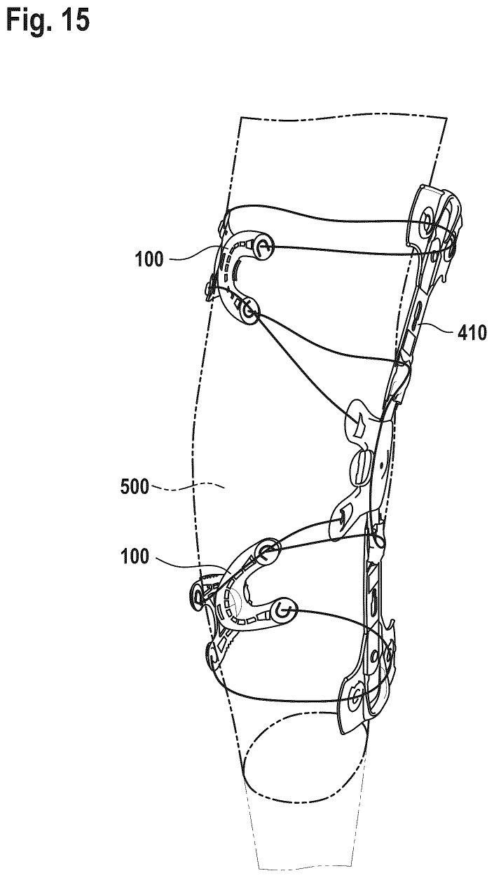

FIG. 15 shows the elbow orthosis from FIG. 14,

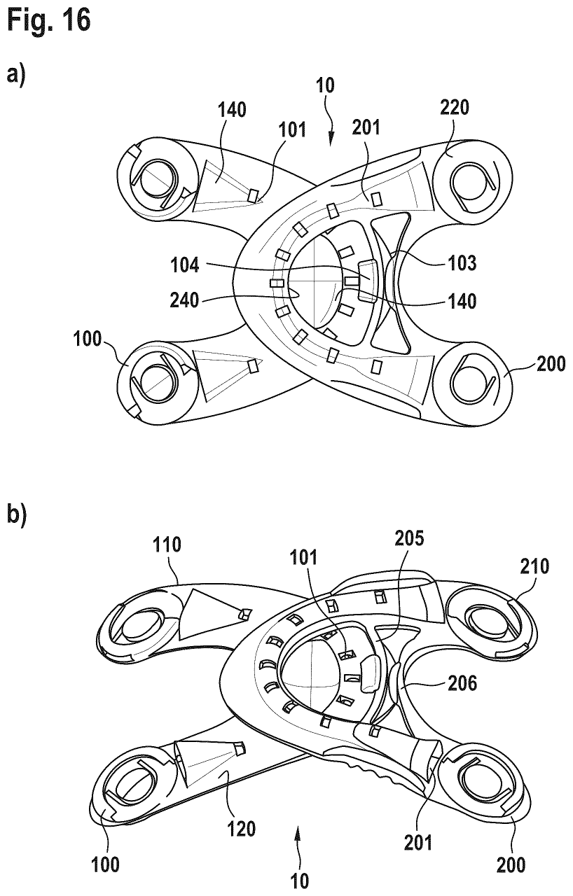

FIG. 16 shows a preferred coupling device for an orthosis according to the invention in the closed state from the front,

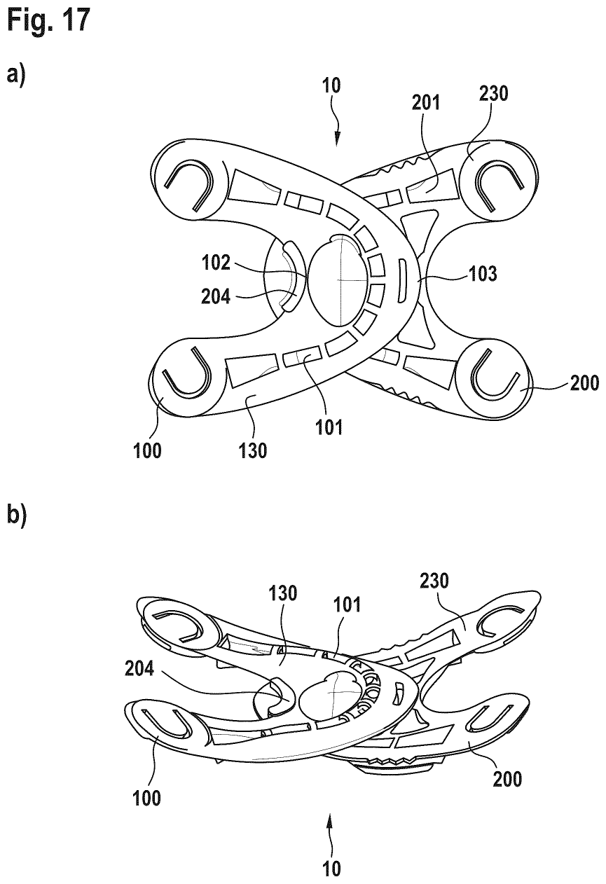

FIG. 17 shows the coupling device from FIG. 16 in the closed state from the rear,

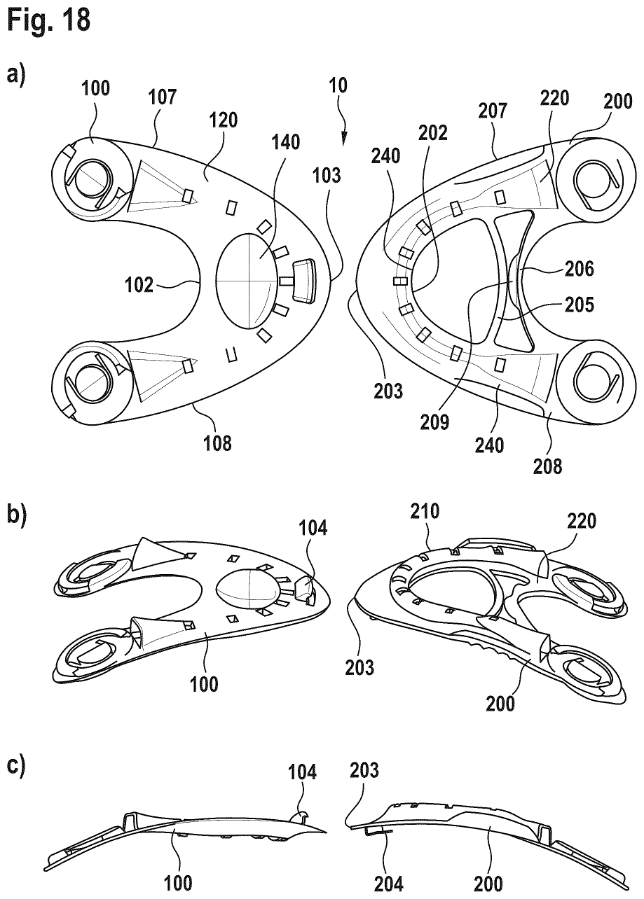

FIG. 18 shows the coupling device from FIG. 16 in the open state from the front,

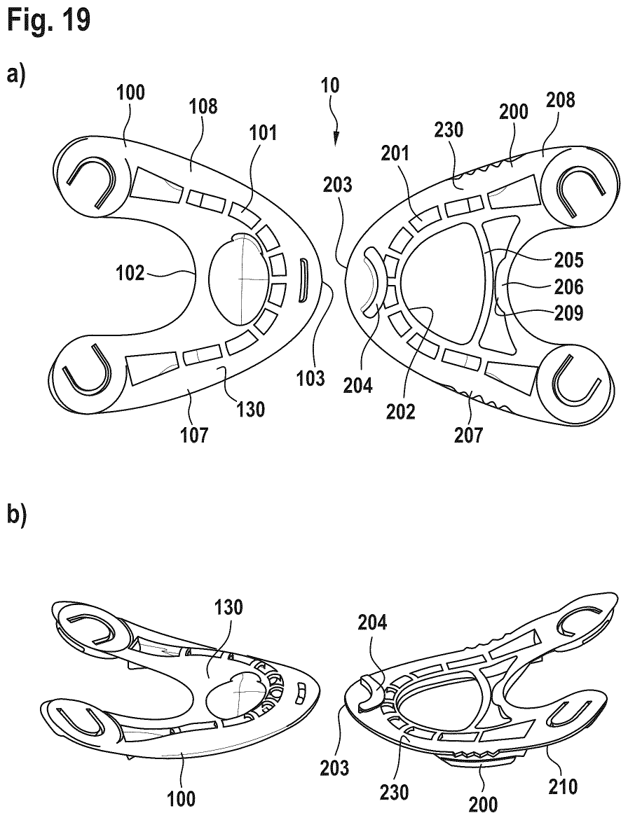

FIG. 19 shows the coupling device from FIG. 16 in the open state from the rear,

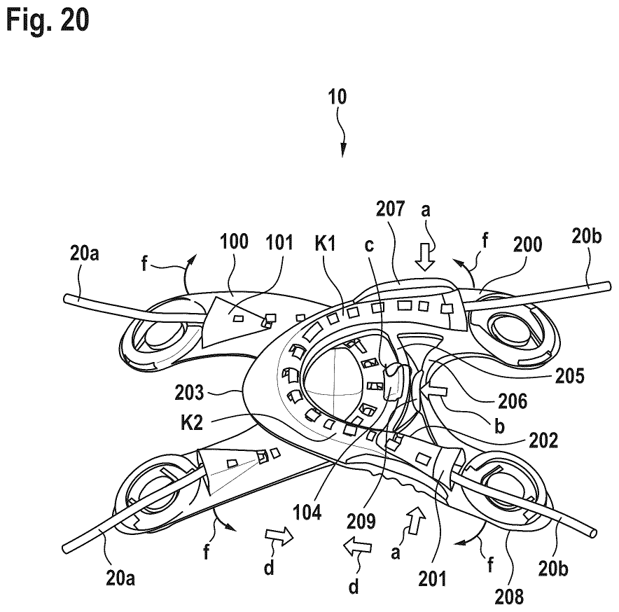

FIG. 20 shows the coupling device from FIG. 16 with details of its use,

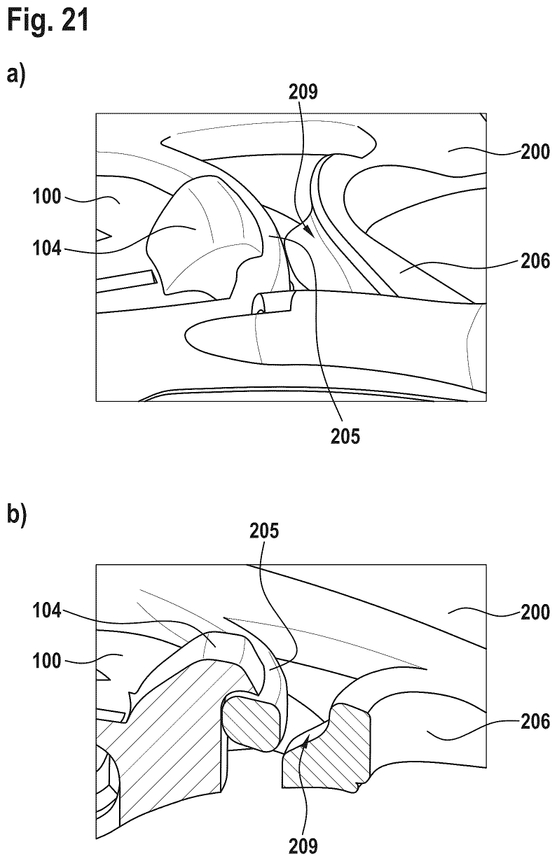

FIG. 21 shows detailed views of the coupling device from FIG. 16.

FIG. 1 shows a limb orthosis according to the invention as a knee orthosis (1000) on a leg (1500) with a thigh (1510), knee area (1520) and lower thigh (1530). A bandage (500) is assigned to the knee orthosis (1000). The knee orthosis comprises a tensioning system (300) consisting of a Bowden cable (320) with a rope (323) largely concealed by a sheathing (321). The Bowden cable (320) runs on a padding (325). The first rope end (322) and the second rope end (324) are connected with a rotary knob (340) for tensioning.

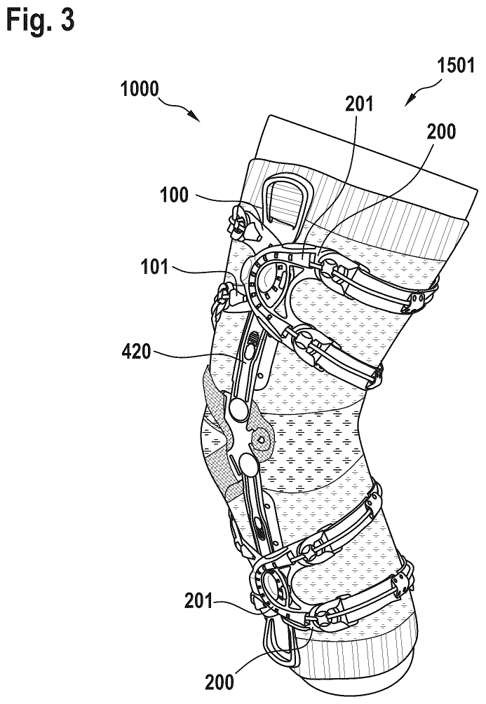

FIGS. 2 and 3 show the knee orthosis (1000) on the inside (1501) of the leg. A second joint splint (420) runs here with a first splint section (421), which is connected via a first joint (424) to a third splint section (423) and a second splint section (422), which is also connected via a joint (425) to the third splint section (423). In the area of the second joint splint (420), there are two coupling devices (10), each consisting of a first coupling element (100) and a second coupling element (200). The two coupling devices (10) simultaneously fulfill two functions, namely, on the one hand, the possibility of reversibly opening and closing the knee orthosis (1000) and, on the other hand, deflecting the Bowden cable (320) via a first guide (10) and a second guide (201).

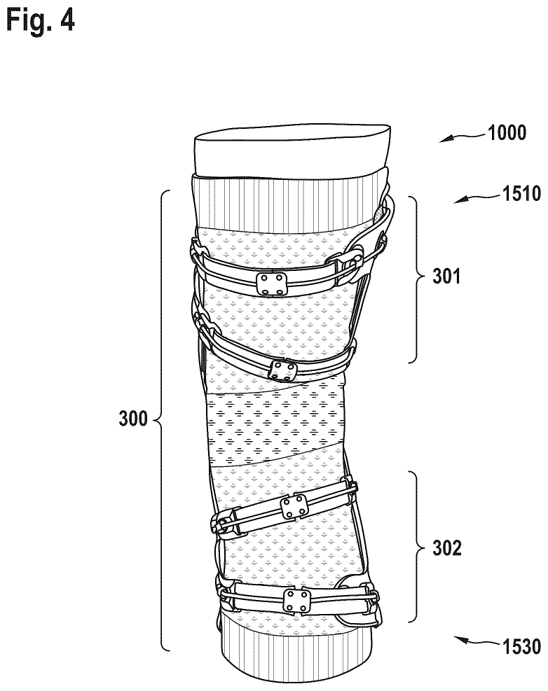

FIG. 4 shows the knee orthosis (1000) on the back side of the leg. It can be seen here how the tensioning system (300) is divided into a first tensioning sub-system (301) on the thigh (1510) and a second tensioning sub-system (302) on the lower thigh (1530), both of which are tensioned.

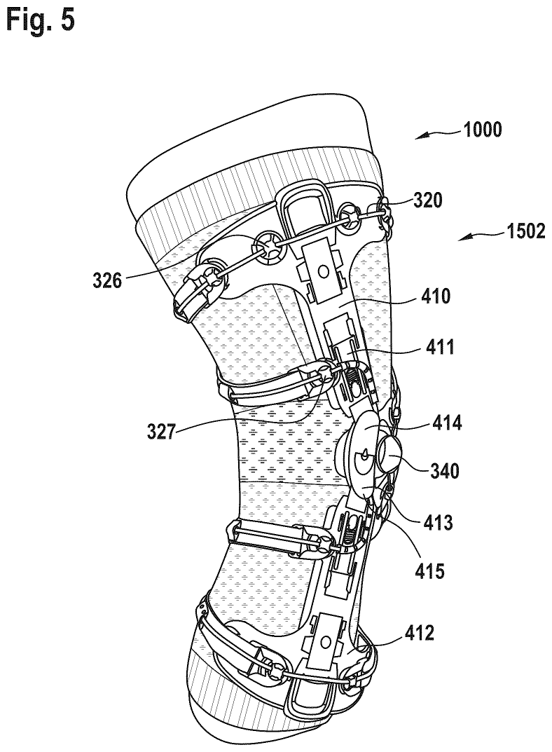

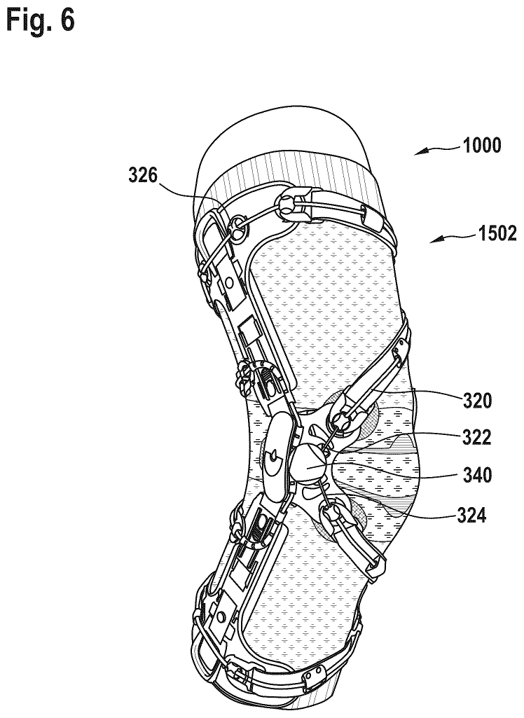

FIG. 5 and FIG. 6 show the knee orthosis (1000) on the outside (1502) of the leg. Here, a first joint splint (410) runs with a first splint section (411), which is connected via a first joint (414) to a third splint section (413), and a second splint section (412), which is also connected via a joint (415) to the third splint section (413). A connection and tension control element designed as a rotary knob (340) is located on the third splint section (413) for tensioning the rope of the Bowden cable (320). The first end (322) and the second end (324) of the rope (323) terminate at the rotary knob (340). The joint splint (410) also comprises rotatably mounted fastening elements (326) and rotatably mounted deflection guides (327) for the Bowden cable (320).

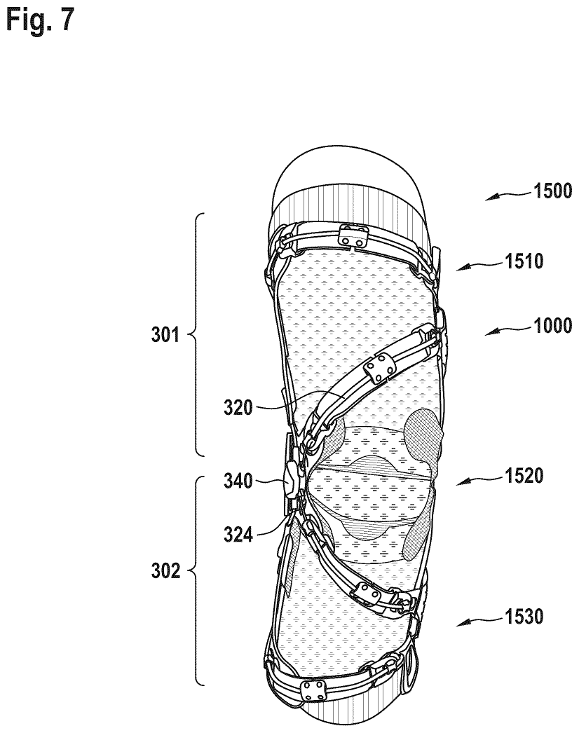

FIG. 7 shows again, as in FIG. 1, the front side of the leg (1500) with the knee orthosis (1000), wherein the division of the tensioning system (300) formed by the Bowden cable (320) into a first tensioning sub-system (301) on the thigh (1510) and a second tensioning sub-system (302) on the lower thigh (1530) can be seen again, wherein the rotary knob (340) is positioned as a tension control element in the knee area (1520).

FIGS. 1 to 7 show, in the order given, the course of the Bowden cable (320) as a tensioning system (300):

The Bowden cable (320) as tensioning element (300) starts with the first end (324) approximately at knee height on the tension control element (340) on the outside of the leg and runs upwards over the thigh (1510) (FIG. 1). The tensioning element then runs to the inside (1501) of the leg, where it is deflected upwards by a guide (101) in the first coupling element (100) of the first coupling system (10) of the first tensioning sub-system (301), whereupon the tensioning element again runs over the thigh (FIG. 2). the tensioning element (300) then slides over the front area of the thigh to the outside of the thigh and runs over the back of the thigh to the inside of the thigh. There, the tensioning element (300) is deflected downward by a guide (201) in the second coupling element (200) of the first coupling system (10) and guided further toward the rear thigh (FIG. 3). Due to the closed first coupling system (10), the tensioning element (300) thus crosses itself twice. The tensioning element (300) runs slightly obliquely downwards on the rear thigh (FIG. 4) to the outside of the leg and is guided there vertically downwards under the knee to the lower thigh and thus to the second tensioning sub-system (302) (FIG. 5). There, the tensioning element (300) runs over the rear side of the lower thigh to the inside of the lower thigh (FIG. 4). There, the tensioning element is deflected downwards by a guide (201) in the second coupling element (200) of the second coupling system (10) of the second tensioning sub-system (302) and guided further towards the rear lower thigh (FIG. 3). The tensioning element (300) runs along the rear lower thigh to the outside of the leg (FIG. 5) and is guided there towards the front lower thigh (FIG. 6) and from there further to the inside of the lower thigh (FIG. 2). There, the tensioning element (300) is deflected upward by the deflection (101) of the first coupling element (100) of the second coupling system (10) of the second tensioning sub-system (302), whereupon the tensioning element (300) runs again over the front side of the lower thigh and ends again with the second end (324) at the tension control element (340) on the outside (FIG. 7). The tension control element (340) can thus be used to tension both ends (322, 324) of the rope (323) of the Bowden cable (320).

FIG. 8 shows the section on the inside (1501) of the thigh (1510). The upper coupling system can be seen with the first coupling element (100) with guide (101) for the Bowden cable (320) and the second coupling element (200) with guide (201) for the Bowden cable (320). The bandage (500) made of a knitted fabric is reversibly connected to the upper, first splint section (421) of the second joint splint (420) via a pocket.

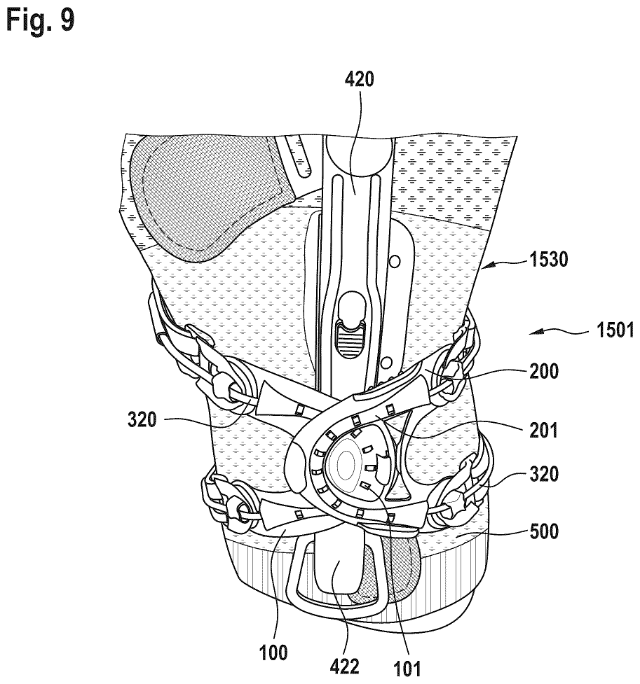

FIG. 9 shows the section on the inside (1501) of the lower thigh (1530). The lower coupling system can be seen with the first coupling element (100) with guide (101) for the Bowden cable (320) and the second coupling element (200) with guide (201) for the Bowden cable (320). The bandage (500) made of a knitted fabric is reversibly connectable to the lower, second splint section (422) of the second joint splint (420) via a pocket.

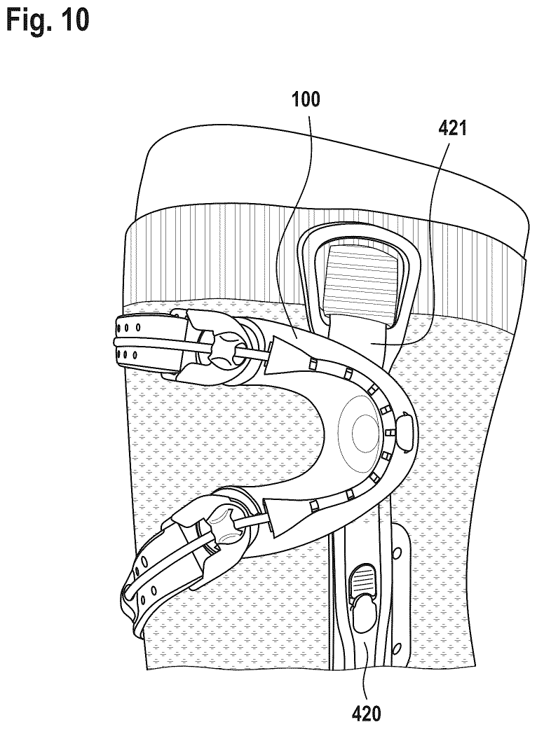

FIG. 10 shows the section from FIG. 8, wherein the upper coupling system is opened and thus the first coupling element (100) is separated from the second coupling element. The first coupling element (100) is connected with the upper, first splint section (421) of the second joint splint (420).

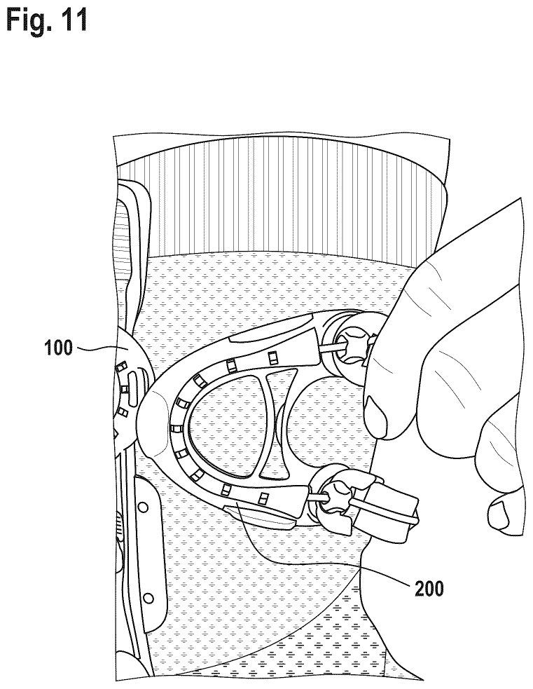

FIG. 11 shows the closing process of the open knee orthosis from FIG. 10. Here, the second coupling element (200) is guided to the first coupling element (100) and connected to it.

FIG. 12 schematically shows a limb orthosis according to the invention as an elbow orthosis (2000) on an arm (2500) with upper arm (2510), elbow area (2520) and forearm (2530). A bandage can be assigned to the elbow orthosis (2000). The elbow orthosis comprises a tensioning system (300) with a rope (323). FIG. 12a) shows the outer arm, FIG. 12b shows the inner arm. A joint splint (410) is located on the outer arm. The two coupling systems (10) for opening and closing the elbow orthosis (2000) are located on the inner arm. Details of the design can be taken from the knee orthosis (1000) in FIGS. 1 to 7.

FIG. 13 schematically shows the elbow orthosis (2000) with rope (323) and coupling systems (100) from FIG. 12 from a different perspective. The joint splint (410) has a first splint section (411) connected to a third splint section (413) via a first joint (414) and a second splint section (412) also connected to the third splint section (413) via a joint (415). A tension control element (not shown) is located on the third splint section (413).

FIG. 14a, FIG. 14b, and FIG. 15 show another embodiment of the elbow orthosis (2000) with coupling systems (100) and joint splint (410) from FIGS. 12 and 13, in which a bandage (500) is assigned to the elbow orthosis (2000). The structure otherwise corresponds to that of FIGS. 12 and 13.

FIG. 16 shows the front side of a preferred embodiment of a coupling device (10) for an orthosis according to the invention with the first coupling element (100) and the second coupling element (200) in a top view (FIG. 16a) and in oblique view (FIG. 16b) and in closed state. Accordingly, the front side (120) of the first coupling element (100) and the front side (220) of the second coupling element (200) can be seen. Both coupling elements (100, 200) are horseshoe-shaped and accordingly have an outer peripheral area (110, 210). The first guide (101) for a tensioning element follows the horseshoe shape of the first coupling element (100) and the second guide (201) for a tensioning element follows the horseshoe shape of the first coupling element (200). The second guide (201) is curved outwards from the front side (220) of the second coupling element (200) and the first guide (101) is flat on the front side (120) of the first coupling element (100) so that the front side (120) of the first coupling element (100) can lie flat on the back side of the second coupling element (200). In an alternative embodiment, the guides (101, 201) can also be dispensed with, particularly if no tensioning element is provided. The first coupling element (100) has a first latching element (104) at its tip (103) of the horseshoe shape, into which the first curved bar element (205) of the second coupling element (200), which spans the concave inner area of the horseshoe shape of the second coupling element (200), is latched.

The second bar element (206) curved in the other direction is located close to the first bar element (205) so that the first bar element (205) is clamped between the first latching element (104) and the second bar element (206). Both the first coupling element (100) comprises handle elements (140) and the second coupling element (200) comprises handle elements (240) which are advantageously formed from the channel-like guides (101, 201).

FIG. 17 shows the back side of the coupling device (10) from FIG. 16 with the first coupling element (100) and the second coupling element (200) in a top view (FIG. 2a) and in an oblique view (FIG. 2b) and in a closed state. The back side (130) of the first coupling element (100) and the back side (230) of the second coupling element (200) can be seen accordingly. The first guide (101) for a tensioning element is designed as a channel with openings and follows the horseshoe shape of the first coupling element (100), and the second guide (201) for a tensioning element is also designed as a channel with openings and follows the horseshoe shape of the first coupling element (200). Here, the first guide (101) is curved outwards from the back side (130) and the second guide (201) is flat on the back side (230) of the second coupling element (200) so that the front side of the first coupling element (100) can lie flat on the back side (230) of the second coupling element (200). The first horseshoe-shaped coupling element (100) has a concave inner edge (102) and an outer tip (103). The concave inner edge (102) of the first coupling element (100) is latched into a second latching element (204) on the back side (230) of the second coupling element (200).

FIG. 18 shows the front side of a coupling device (10) from FIG. 16 with the first coupling element (100) and the second coupling element (200) in a top view (FIG. 3a) and in an oblique view (FIG. 3b) and in a side view (FIG. 3c) in the open state. The front side (120) of the first coupling element (100) and the front side (220) of the second coupling element (200) can be seen accordingly. Both coupling elements (100, 200) are horseshoe-shaped and accordingly have an outer peripheral area (210) and a first arm (107, 207) and a second arm (108, 208).

The first coupling element (100) has a concave inner edge (102) and, at its tip (103) of the horseshoe shape, a first latching element (104) into which the first curved bar element (205) of the second coupling element (200), which spans the concave inner region of the horseshoe shape of the second coupling element (200), can be latched. The second bar element (206) curved in the other direction is located close to the first bar element (205) so that the first bar element (205) can be clamped between the first latching element (104) and the second bar element (206). The bar element (206) has an oblique flattening (209) in the direction of the first bar element (205), which can slide under the first bar element (205). The second coupling element (200) has a concave inner edge (202) and, at its tip (203) of the horseshoe shape on the underside, a second latching element (204) into which the concave inner edge (102) of the first coupling element (100) can be latched. Both the first coupling element (100) and the second coupling element (200) comprise handle elements (140, 240).

FIG. 19 shows the back side of the coupling device (10) from FIG. 16 with the first horseshoe-shaped coupling element (100) with first (107) and second (108) arms and the second horseshoe-shaped coupling element (200) with first (207) and second (208) arms and the peripheral area (210) in a top view (FIG. 4a) and in an oblique view (FIG. 4b) and in the open state. Accordingly, the back side (130) of the first coupling element (100) and the back side (230) of the second coupling element (200) can be seen. The first guide (101) for a tensioning element is designed as a channel with openings and follows the horseshoe shape of the first coupling element (100), and the second guide (201) for a tensioning element is also designed as a channel with openings and follows the horseshoe shape of the first coupling element (200). Here, the first guide (101) is curved outwards from the back side (130) and the second guide (201) is flat on the back side (230) of the second coupling element (200) so that the front side of the first coupling element (100) can lie flat on the back side (230) of the second coupling element (200). The first horseshoe-shaped coupling element (100) has a concave inner edge (102) and an outer tip (103). The concave inner edge (102) of the first coupling element (100) can be latched into a second latching element (204) on the back side (230) at the tip (203) of the second coupling element (200). The second coupling element (200) has a concave inner edge (202), the space between which is spanned by the first bar element (205) and the second bar element (206) with the oblique flattening (209).

FIG. 20 shows the coupling device (10) from FIG. 16 with the first coupling element (100) and the second coupling element (200) schematically during use, particularly before/during the opening of the coupling device (10). A first tensioning element (20a), for example a rope or a Bowden cable, runs in the first guide (101), and a second tensioning element (20b), for example also a rope or a Bowden cable, runs in the second guide (201). Preferably, the first tensioning element (20a) and the second tensioning element (20b) can be partial sections of a single tensioning element. Both the guides (101, 201) and the tensioning elements (20a, 20b) cross each other twice in an advantageous manner when the coupling device (10) is in the closed state, namely at a first crossing point (K1) and at a second crossing point (K2). An advantage of the coupling device (10) according to the invention is that the two coupling elements (100, 200) are rotatably mounted relative to each other, as indicated by the directions of movement (f).

The coupling device (10) is opened by compressing the second horseshoe-shaped coupling element (200) at the arms (207, 208), as indicated by the arrows (a), whereby the second bar element (206) spanning the concave inner edge (202) of the second coupling element (200) is pushed with its oblique flattening (209) in the direction of the tip (203) of the second horseshoe-shaped coupling element (200) and in the direction of the first coupling element (100), as indicated by the arrow (b), and whereby the second bar element (206) spanning the concave inner edge (202) of the second coupling element (200) lifts the first bar element (205) out of the first latching element (104) with its oblique flattening (209), as indicated by the arrow (c), so that the two coupling elements (100, 200) are pushed against each other and can thus be separated from each other, as indicated by the arrows (d).

FIG. 21 shows two detailed views of the front side of the coupling device from FIG. 16 with the first coupling element (100) and the second coupling element (200) in an oblique view (FIG. 21a) and in cross-section (FIG. 21b) in the closed state. The first coupling element (100) has a first latching element (104) at its tip, into which the first curved bar element (205) of the second coupling element (200), which spans the concave inner area of the horseshoe shape of the second coupling element (200), is latched. The second bar element (206), which is curved in the other direction, lies close to the first bar element (205) so that the first bar element (205) is clamped between the first latching element (104) and the second bar element (206). The bar element (206) has an oblique flattening (209) in the direction of the first bar element (205), which can slide under the first bar element (205).

### LIST OF REFERENCE SIGNS

10 coupling device
20a tensioning element
20b tensioning element
100 first coupling element
101 first guide
102 concave inner edge of the first coupling element
103 tip of the horseshoe shape of the first coupling element
104 first latching element of the first coupling element
107 first arm of the horseshoe-shaped first coupling element
108 second arm of the horseshoe-shaped first coupling element
110 peripheral area of the first coupling element
120 front side of the first coupling element
130 back side of the first coupling element
140 handle element of the first coupling element
200 second coupling element
201 second guide
202 concave inner edge of the second coupling element
203 tip of the horseshoe shape of the second coupling element
204 second latching element of the second coupling element
205 first bar element
206 second bar element
207 first arm of the horseshoe-shaped second coupling element
208 second arm of the horseshoe-shaped second coupling element
209 oblique flattening on the second bar element
210 peripheral area of the second coupling element
220 front side of the second coupling element
230 back side of the second coupling element
240 handle element of the second coupling element
300 tensioning system
301 first tensioning sub-system
302 second tensioning sub-system
321 sheathing
322 first end of the Bowden cable rope
323 rope
324 second end of the Bowden cable rope
325 padding
326 rotatably mounted fastening element
327 rotatably mounted deflection guide
340 rotary knob as connection and tension control element
410 first joint splint
411 first splint section
412 second splint section
413 third splint section
414 first joint
415 second joint
420 second joint splint
421 first splint section
422 second splint section
423 third splint section
424 first joint
425 second joint
500 bandage
1000 knee orthosis
1500 leg
1501 inside of the leg
1502 outside of the leg
1510 thigh
1520 knee area
1530 lower thigh
2000 elbow orthosis
2500 arm
2501 outside of the arm
2502 inside of the arm
2510 upper arm
2530 forearm
a first step of opening
b second step of opening
c third step of opening
d fourth step of opening
f rotational movement
K1 first crossing point
K2 second crossing point

1. A limb orthosis, limb bandage or sports aid device, comprising a tensioning system consisting of a first tensioning sub-system and a second tensioning sub-system, wherein the first tensioning sub-system and the second tensioning sub-system are each reversibly closable by a coupling device, wherein each coupling device comprises a first coupling element and a second coupling element, wherein the first coupling element is reversibly connectable to the second coupling element via a latching system, wherein the first coupling element comprises a first guide for a tensioning element and the second coupling element comprises a second guide for a tensioning element, wherein the tensioning element runs in the first guide and the tensioning element runs in the second guide and wherein, in a connected state of the first and second coupling elements, the first guide and the second guide cross each other twice.

2. The limb orthosis, limb bandage or sports aid device according to claim 1, wherein the first tensioning sub-system and the second tensioning sub-system are formed from a continuous tensioning element.

3. The limb orthosis, limb bandage or sports aid device according to claim 1, wherein the first tensioning sub-system is formed from a first tensioning element and the second tensioning sub-system is formed from a second tensioning element.

4. The limb orthosis, limb bandage or sports aid device according to claim 2, wherein the at least one tensioning element is a rope, a wire, a flat band or a Bowden cable.

5. A limb orthosis, limb bandage or sports aid device according to claim 4, wherein the at least one tensioning element comprises a rope or a wire, wherein the rope or the wire runs at least partially in a sheathing, wherein the sheathing is displaceably mounted on the limb orthosis.

6. The limb orthosis, limb bandage or sports aid device of claim 1, wherein the first coupling element comprises a front side and a back side and comprises a horseshoe shape with a concave inner edge, and wherein the second coupling element comprises a front side and a back side and comprises a horseshoe shape with a concave inner edge, wherein the first coupling element comprises a first latching element in an area of a tip of the horseshoe shape on the front side and the second coupling element comprises a second latching element in the area of the tip of the horseshoe shape on the back side and wherein the second horseshoe-shaped coupling element comprises a first bar element spanning the concave inner edge of the second coupling element, wherein, in the connected state of the two coupling elements, the first latching element of the first coupling element engages in the first bar element of the second coupling element and the second latching element of the second coupling element engages in the concave inner edge of the first coupling element.

7. The limb orthosis, limb bandage or sports aid device according to claim 1, wherein the limb orthosis comprises rotatably mounted deflection guides in which the at least one tensioning element is deflected.

8. The limb orthosis, limb bandage or sports aid device according to claim 1, wherein the limb orthosis or bandage is a knee orthosis or bandage or the sports aid device is a sports aid device for the knee.

9. The limb orthosis, limb bandage or sports aid device according to claim 1, wherein, when the limb orthosis is applied, the first tensioning sub-system and/or the second tensioning sub-system of the tensioning system (are guided around the limb, in particular a leg, in such a way that the first tensioning sub-system and/or the second tensioning sub-system of the tensioning system each enclose the limb.

10. The limb orthosis, limb bandage or sports aid device according to claim 2, wherein the limb orthosis comprises a connection element to which the at least one tensioning element, in particular the continuous single tensioning element is fastened.

11. The limb orthosis, limb bandage or sports aid device according to claim 10, wherein the tensioning element is fastened with both ends to the connection element.

12. The limb orthosis, limb bandage or sports aid device according to claim 10, wherein the connection element comprises a tension control element for tensioning the at least one tensioning element.

13. The limb orthosis, limb bandage or sports aid device according to claim 1, wherein the limb orthosis, limb bandage or sports aid device comprises at least one joint splint.

14. The limb orthosis, limb bandage or sports aid device according to claim 13, wherein the at least one joint splint comprises a first splint section, a second splint section and a third splint section, wherein the first splint section and the second splint section are each connected to the third splint section by a joint.

15. The limb orthosis, limb bandage or sports aid device according to claim 14, wherein the connection element is fastened to the third splint section of the at least one joint splint.

16. The limb orthosis, limb bandage or sports aid device according to claim 3, wherein the limb orthosis comprises a connection element to which the at least one tensioning element, in the second tensioning element, are fastened.

17. [symbol]
a tensioning system including a first tensioning sub-system and a second tensioning sub-system, wherein the first tensioning sub-system and the second tensioning sub-system are each reversibly closable by a coupling device;
the coupling device comprising a first coupling element and a second coupling element, the first coupling element being reversibly connectable to the second coupling element via a latching system;
the first coupling element including a first guide for a tensioning element and the second coupling element including a second guide for a tensioning element;
and wherein a single continuous tensioning element is routed through both the first guide and the second guide, and wherein, in a connected configuration of the two coupling elements, the first guide and the second guide cross each other at least once.

18. The limb support system of claim 17, wherein, in a connected configuration of the two coupling elements, the first guide and the second guide cross each other at least once.

19. A limb support system comprising:
a tensioning system including a first tensioning sub-system and a second tensioning sub-system;
wherein the first tensioning sub-system and the second tensioning sub-system are each reversibly closable by a coupling device, each coupling device comprising a first coupling element and a second coupling element;
the first coupling element reversibly connectable to the second coupling element via a latching system, the first coupling element including a first guide for a tensioning element and the second coupling element including a second guide for a tensioning element; and
wherein the tensioning element runs in the first guide and the tensioning element runs in the second guide, and wherein, in a connected configuration of the two coupling elements, the first guide and the second guide cross each other twice.
